SEQUENCE LISTING (6 sequences)

SEQ ID NO: 1 - RNA, 1469 nt, Bifidobacterium animalis
  source 1..1469: mol_type = rRNA
atggctcaggatgaacgctggcggcgtgcttaacacatgcaagtcgaacgggatccctggcagcttgctgtcggggtgagagtggcgaacgggtgagtaatgcgtgaccaacctgccctgtgcaccggaatagctcctggaaacgggtggtaataccggatgctccgctccatcgcatggtggggtgggaaatgcttttgcggcatgggatggggtcgcgtcctatcagcttgttggcggggtgatggcccaccaaggcgttgacgggtagccggcctgagagggtgaccggccacattgggactgagatacggcccagactcctacgggaggcagcagtggggaatattgcacaatgggcgcaagcctgatgcagcgacgccgcgtgcgggatggaggccttcgggttgtaaaccgcttttgttcaagggcaaggcacggtttcggccgtgttgagtggattgttcgaataagcaccggctaactacgtgccagcagccgcggtaatacgtagggtgcgagcgttatccggatttattgggcgtaaagggctcgtaggcggttcgtcgcgtccggtgtgaaagtccatcgcctaacggtggatctgcgccgggtacgggcgggctggagtgcggtaggggagactggaattcccggtgtaacggtggaatgtgtagatatcgggaagaacaccaatggcgaaggcaggtctctgggccgtcactgacgctgaggagcgaaagcgtggggagcgaacaggattagataccctggtagtccacgccgtaaacggtggatgctggatgtggggccctttccacgggtcccgtgtcggagccaacgcgttaagcatcccgcctggggagtacggccgcaaggctaaaactcaaagaaattgacgggggcccgcacaagcggcggagcatgcggattaattcgatgcaacgcgaagaaccttacctgggcttgacatgtgccggatcgccgtggagacacggtttcccttcggggccggttcacaggtggtgcatggtcgtcgtcagctcgtgtcgtgagatgttgggttaagtcccgcaacgagcgcaaccctcgccgcatgttgccagcgggtgatgccgggaactcatgtgggaccgccggggtcaactcggaggaaggtggggatgacgtcagatcatcatgccccttacgtccagggcttcacgcatgctacaatggccggtacaacgcggtgcgacacggtgacgtggggcggatcgctgaaaaccggtctcagttcggatcgcagtctgcaactcgactgcgtgaaggcggagtcgctagtaatcgcggatcagcaacgccgcggtgaatgcgttcccgggccttgtacacaccgcccgtcaagtcatgaaagtgggtagcacccgaagccggtggcccgacccttgtggggggagccgtctaaagtgagactcgtgattgggact

SEQ ID NO: 2 - RNA, 1468 nt, Bifidobacterium animalis
  source 1..1468: mol_type = rRNA
atggctcaggatgaacgctggcggcgtgcttaacacatgcaagtcgaacgggatccctggcagcttgctgtcggggtgagagtggcgaacgggtgagtaatgcgtgaccaacctgccctgtgcaccggaatagctcctggaaacgggtggtaataccggatgctccgctccatcgcatggtggggtgggaaatgcttttgcggcatgggatggggtcgcgtcctatcagcttgttggcggggtgatggcccaccaaggcgttgacgggtagccggcctgagagggtgaccggccacattgggactgagatacggcccagactcctacgggaggcagcagtggggaatattgcacaatgggcgcaagcctgatgcagcgacgccgcgtgcgggatggaggccttcgggttgtaaaccgcttttgttcaagggcaaggcacggtttcggccgtgttgagtggattgttcgaataagcaccggctaactacgtgccagcagccgcggtaatacgtagggtgcgagcgttatccggatttattgggcgtaaagggctcgtaggcggttcgtcgcgtccggtgtgaaagtccatcgcctaacggtggatctgcgccgggtacgggcgggctggagtgcggtaggggagactggaattcccggtgtaacggtggaatgtgtagatatcgggaagaacaccaatggcgaaggcaggtctctgggccgtcactgacgctgaggagcgaaagcgtggggagcgaacaggattagataccctggtagtccacgccgtaaacggtggatgctggatgtggggccctttccacgggtcccgtgtcggagccaacgcgttaagcatcccgcctggggagtacggccgcaaggctaaaactcaaagaaattgacgggggcccgcacaagcggcggagcatgcggattaattcgatgcaacgcgaagaaccttacctgggcttgacatgtgccggatcgccgtggagacacggtttcccttcggggccggttcacaggtggtgcatggtcgtcgtcagctcgtgtcgtgagatgttgggttaagtcccgcaacgagcgcaaccctcgccgcatgttgccagcgggtgatgccgggaactcatgtgggaccgccggggtcaactcggaggaaggtggggatgacgtcagatcatcatgccccttacgtccagggcttcacgcatgctacaatggccggtacaacgcggtgcgacacggtgacgtggggcggatcgctgaaaaccggtctcagttcggatcgcagtctgcaactcgactgcgtgaaggcggagtcgctagtaatcgcggatcagcaacgccgcggtgaatgcgttcccgggccttgtacacaccgcccgtcaagtcatgaaagtgggtagcacccgaagccggtggcccgacccttgtggggggagccgtctaagtgagactcgtgattgggact

SEQ ID NO: 3 - RNA, 1492 nt, Lactobacillus paracasei
  source 1..1492: mol_type = rRNA
gatgaacgctggcggcgtgcctaatacatgcaagtcgaacgagttctcgttgatgatcggtgcttgcaccgagattcaacatggaacgagtggcggacgggtgagtaacacgtgggtaacctgcccttaagtgggggataacatttggaaacagatgctaataccgcatagatccaagaaccgcatggttcttggctgaaagatggcgtaagctatcgcttttggatggacccgcggcgtattagctagttggtgaggtaatggctcaccaaggcgatgatacgtagccgaactgagaggttgatcggccacattgggactgagacacggcccaaactcctacgggaggcagcagtagggaatcttccacaatggacgcaagtctgatggagcaacgccgcgtgagtgaagaaggctttcgggtcgtaaaactctgttgttggagaagaatggtcggcagagtaactgttgtcggcgtgacggtatccaaccagaaagccacggctaactacgtgccagcagccgcggtaatacgtaggtggcaagcgttatccggatttattgggcgtaaagcgagcgcaggcggttttttaagtctgatgtgaaagccctcggcttaaccgaggaagcgcatcggaaactgggaaacttgagtgcagaagaggacagtggaactccatgtgtagcggtgaaatgcgtagatatatggaagaacaccagtggcgaaggcggctgtctggtctgtaactgacgctgaggctcgaaagcatgggtagcgaacaggattagataccctggtagtccatgccgtaaacgatgaatgctaggtgttggagggtttccgcccttcagtgccgcagctaacgcattaagcattccgcctggggagtacgaccgcaaggttgaaactcaaaggaattgacgggggcccgcacaagcggtggagcatgtggtttaattcgaagcaacgcgaagaaccttaccaggtcttgacatcttttgatcacctgagagatcaggtttccccttcgggggcaaaatgacaggtggtgcatggttgtcgtcagctcgtgtcgtgagatgttgggttaagtcccgcaacgagcgcaacccttatgactagttgccagcatttagttgggcactctagtaagactgccggtgacaaaccggaggaaggtggggatgacgtcaaatcatcatgccccttatgacctgggctacacacgtgctacaatggatggtacaacgagttgcgagaccgcgaggtcaagctaatctcttaaagccattctcagttcggactgtaggctgcaactcgcctacacgaagtcggaatcgctagtaatcgcggatcagcacgccgcggtgaatacgttcccgggccttgtacacaccgcccgtcacaccatgagagtttgtaacacccgaagccggtggcgtaacccttttagggagcgagccgtctaaagtgggacaaatgattagggtg

SEQ ID NO: 4 - RNA, 1493 nt, Lactobacillus gasseri
  source 1..1493: mol_type = rRNA
gacgaacgctggcggcgtgcctaatacatgcaagtcgagcgagcttgcctagatgaatttggtgcttgcaccaaatgaaactagatacaagcgagcggcggacgggtgagtaacacgtgggtaacctgcccaagagactgggataacacctggaaacagatgctaataccggataacaacactagacgcatgtctagagtttaaaagatggttctgctatcactcttggatggacctgcggtgcattagctagttggtaaggtaacggcttaccaaggcaatgatgcatagccgagttgagagactgatcggccacattgggactgagacacggcccaaactcctacgggaggcagcagtagggaatcttccacaatggacgcaagtctgatggagcaacgccgcgtgagtgaagaagggtttcggctcgtaaagctctgttggtagtgaagaaagatagaggtagtaactggcctttatttgacggtaattacttagaaagtcacggctaactacgtgccagcagccgcggtaatacgtaggtggcaagcgttgtccggatttattgggcgtaaagcgagtgcaggcggttcaataagtctgatgtgaaagccttcggctcaaccggagaattgcatcagaaactgttgaacttgagtgcagaagaggagagtggaactccatgtgtagcggtggaatgcgtagatatatggaagaacaccagtggcgaaggcggctctctggtctgcaactgacgctgaggctcgaaagcatgggtagcgaacaggattagataccctggtagtccatgccgtaaacgatgagtgctaagtgttgggaggtttccgcctctcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatccagtgcaaacctaagagattaggagttcccttcggggacgctgagacaggtggtgcatggctgtcgtcagctcgtgtcgtgagatgttgggttaagtcccgcaacgagcgcaacccttgtcattagttgccatcattaagttgggcactctaatgagactgccggtgacaaaccggaggaaggtggggatgacgtcaagtcatcatgccccttatgacctgggctacacacgtgctacaatggacggtacaacgagaagcgaacctgcgaaggcaagcggatctctgaaagccgttctcagttcggactgtaggctgcaactcgcctacacgaagctggaatcgctagtaatcgcggatcagcacgccgcggtgaatacgttcccgggccttgtacacaccgcccgtcacaccatgagagtctgtaacacccaaagccggtgggataacctttataggagtcagccgtctaagtagacagatgattagggtg

SEQ ID NO: 5 - DNA, 19 bp, synthetic construct
  source 1..19: mol_type = other DNA
agagtttgatcmtggctca

SEQ ID NO: 6 - DNA, 22 bp, synthetic construct
  source 1..22: mol_type = other DNA
tacggytaccttgttacgactt

(19) United States
(12) Patent Application Publication
(10) Pub. No.: US 2026/0275273 A1
(43) Pub. Date: Sep. 17, 2026

(54) NOVEL BIFIDOBACTERIUM AND/OR LACTOBACILLUS STRAIN OR COMBINATION THEREOF AND USES THEREOF

(71) Applicant: KOREA RESEARCH INSTITUTE OF BIOSCIENCE AND BIOTECHNOLOGY, Daejeon (KR)

(72) Inventors: Doo-Sang PARK, Daejeon (KR); Kwang-Pyo LEE, Daejeon (KR); Yong Ryoul YANG, Daejeon (KR); Minju KIM, Daejeon (KR); Seung Hyun KIM, Daejeon (KR); Jaewon YANG, Daejeon (KR)

(73) Assignee: KOREA RESEARCH INSTITUTE OF BIOSCIENCE AND BIOTECHNOLOGY, Daejeon (KR)

(21) Appl. No.: 19/168,554

(22) PCT Filed: Mar. 29, 2024
(86) PCT No.: PCT/KR2024/004017

§ 371 Date: Sep. 24, 2025

(30) Foreign Application Priority Data
Mar. 29, 2023 (KR) 10-2023-0041564

(51) Int. Cl. C12N 1/205; A61K 35/00; A61P 21/00; C12R 1/225

(52) U.S. Cl. CPC C12N 1/205; A61P 21/00; A61K 2035/115; C12R 2001/225

(57) ABSTRACT

The present invention relates to two novel Bifidobacterium animalis spp. lactis strains, Lactobacillus paracasei strains, Lactobacillus gasseri strains, or their combinations and the use thereof, which increase the activity of AMP-activated protein kinase (AMPK) protein present in muscle cells, thereby increasing mitochondrial synthesis and enhancing muscle exercise performance. Accordingly, the strains may be advantageously used to improve muscle function, or for the prevention, improvement, or treatment of muscle diseases, as pharmaceuticals, health functional foods, and foods.

### TECHNICAL FIELD

### Cross-Reference to Related Applications

This application claims priority from Korean Patent Application No. 10-2023-0041564, filed on Mar. 29, 2023, the disclosure of which is incorporated by reference herein.

### TECHNICAL FIELD

The present invention relates to novel Bifidobacterium and/or Lactobacillus strains, or their combinations, and the use thereof.

### BACKGROUND ART

Decrease in muscle mass leads to serious disability and secondary geriatric diseases, resulting in economic and social losses. In particular, from the late 50s, sarcopenia patients begin to occur, and sarcopenia is the disease with the highest prevalence, affecting 40 to 50% of the population aged 65 and older. Aging-related muscle loss not only causes disability and gait disturbances, but also leads to various secondary geriatric diseases such as diabetes, hypertension, and cardiovascular disease, making independent living impossible and requiring long-term care, shortening healthy life expectancy. Due to its importance, the World Health Organization (WHO) assigned a disease code to sarcopenia in 2016. Global pharmaceutical companies are developing a sarcopenia therapeutic agent, but to date, no FDA-approved drugs have been developed.

AMP-activated protein kinase (AMPK) is an enzyme that plays a crucial role in maintaining cellular energy homeostasis. The AMPK is composed of three subunits of α, β, and γ and is a protein complex that is evolutionarily well-conserved from yeast to humans. It is well known that increasing AMPK protein activity in muscle increases mitochondrial biogenesis and increases muscle motor function.

There is a growing need for functional foods or pharmaceutical compositions that enhance AMPK activity in muscle to improve muscle function in muscle diseases, including sarcopenia.

### DISCLOSURE OF THE INVENTION

### Technical Problem

An objective of the present invention is to provide two novel Bifidobacterium animalis spp. lactis strain.

Another objective of the present invention is to provide a novel Lactobacillus paracasei strain.

objective of the present invention is to provide a novel Lactobacillus gasseri strain.

Another objective of the present invention is to provide a composition comprising the novel strain or a combination of strains.

Another objective of the present invention is to provide a use for the composition.

### Technical Solution

To achieve the above object, an aspect of the present invention provides Bifidobacterium animalis spp. lactis DS109-B11 strain, deposited under accession No. KCTC 15297BP.

To achieve the above object, another aspect of the present invention provides Bifidobacterium animalis spp. lactis DS108-B7 strain, deposited under accession No. KCTC 15298BP.

In addition, to achieve the above object, another aspect of the present invention provides Lactobacillus paracasei DS108-B10 strain, deposited under accession No. KCTC 15343BP.

In addition, to achieve the above object, another aspect of the present invention provides Lactobacillus gasseri DS108-B6 strain, deposited under accession No. KCTC 15299BP.

In addition, to achieve the above object, another aspect of the present invention provides a composition comprising at least one selected from the group consisting of the strain, a combination of the strains, a culture medium of the strain or a combination of strains, a concentrate of the culture medium, a dried product of the culture medium, and an extract of the culture medium.

In addition, to achieve the above object, another aspect of the present invention provides a health functional food composition for improving muscle function comprising at least one selected from the group consisting of the strain, a combination of the strains, a culture medium of the strain or the combination of strains, a concentrate of the culture medium, a dried product of the culture medium, and an extract of the culture medium.

In addition, to achieve the above object, another aspect of the present invention provides a pharmaceutical composition for treatment or prevention of muscle diseases comprising at least one selected from the group consisting of the strain, a combination of the strains, a culture medium of the strain or the combination of strains, a concentrate of the culture medium, a dried product of the culture medium, and an extract of the culture medium.

### Advantageous Effects

The present invention relates to novel Bifidobacterium animalis spp. lactis strains, Lactobacillus paracasei strain, Lactobacillus strain, gasseri or strains selected from combinations thereof, which increase the activity of AMP-activated protein kinase (AMPK) protein present in muscle cells, thereby increasing mitochondrial synthesis and enhancing muscle exercise performance. Accordingly, the strains may be advantageously used to improve muscle function, or for the prevention, improvement, or treatment of muscle diseases, as pharmaceuticals, health functional foods, foods, or feeds.

However, the effects of the present invention are not limited to the effects described above, and other effects not mentioned will be clearly understood by those skilled in the art from the following description.

### BRIEF DESCRIPTION OF THE DRAWINGS

FIG. 1 illustrates the experimental results of Western blot analysis to determine the amount of AMPK protein activated by the novel Bifidobacterium animalis spp. lactis strain (DS109-B11) and Lactobacillus gasseri strain (DS108-B6) of the present invention in a C2C12 muscle cell line.

FIG. 2 illustrates the experimental results of quantifying the amount of AMPK protein activated by two novel Bifidobacterium animalis spp. lactis strains (DS109-B11 and DS108-B7), Lactobacillus paracasei strain (DS108-B10), and Lactobacillus gasseri strain (DS108-B6) of the present invention in the C2C12 muscle cell line.

FIG. 3 illustrates the experimental results confirming the improved muscle cell differentiation effect of the novel Bifidobacterium animalis spp. lactis (DS109-B11) of the present invention in a C2C12 muscle cell line.

FIG. 4 illustrates the experimental results confirming the improved muscle cell differentiation effect of the novel Lactobacillus gasseri strain (DS108-B6) of the present invention in the C2C12 muscle cell line.

FIG. 5 illustrates the experimental results confirming increases in grip strength, muscle mass relative to body weight, and exercise capacity in young and aged mice models after treatment with Bifidobacterium animalis spp. lactis DS109-B11 strain of the present invention.

FIG. 6 illustrates the experimental results verifying AMPK protein activity in aged mouse muscles treated with Bifidobacterium animalis spp. lactis DS109-B11 strain of the present invention.

FIG. 7 illustrates the experimental results confirming an increase in mitochondrial quantity by confirming the levels of representative electron transport chain-related proteins of mitochondrial complexes I, II, III, IV, and V in aged mouse muscles treated with Bifidobacterium animalis spp. lactis DS109-B11 strain of the present invention.

### MODE FOR CARRYING OUT THE INVENTION

Hereinafter, the present invention will be described in detail.

1. Two Novel Bifidobacterium animalis Spp. Lactis Strains

One aspect of the present invention provides a novel Bifidobacterium animalis spp. lactis DS109-B11 strain.

The novel Bifidobacterium animalis spp. lactis strain of the present invention may comprise 16S rRNA having the base sequence of SEQ ID NO: 1, and may be, in particular, a Bifidobacterium animalis spp. lactis DS109-B11 strain, deposited under accession No. KCTC 15297BP with the Korean Collection for Type Cultures (KCTC), on Jan. 18, 2023.

Another aspect of the present invention provides a novel Bifidobacterium animalis spp. lactis DS108-B7 strain.

The novel Bifidobacterium animalis spp. lactis strain of the present invention may comprise 16S rRNA having the base sequence of SEQ ID NO: 2, and may be, in particular, the Bifidobacterium animalis spp. lactis DS108-B7 strain deposited under accession No. KCTC 15298BP with the Korean Collection for Type Cultures (KCTC), on Jan. 18, 2023.

The two novel Bifidobacterium animalis spp. lactis strains of the present invention may be strains capable of inhabiting the intestines of humans or non-human animals, and may have various effects on the health of humans or non-human animals while inhabiting the intestines. The two novel Bifidobacterium animalis spp. lactis strains of the present invention may be safe microorganisms that do not exhibit toxicity or cause disease in humans or non-human animals. The two novel Bifidobacterium animalis spp. lactis strains may act as beneficial bacteria in the intestines that contribute to the health of humans or non-human animals, and may function as probiotic microorganisms that improve muscle function.

The probiotics are live microorganisms that provide health benefits and play a crucial role in suppressing harmful bacteria and increasing beneficial bacteria in intestinal flora. To be recognized as the probiotics, the probiotics should survive gastric acid and bile acids, reach the small and large intestines, proliferate and establish in the intestines, exhibit beneficial effects within the intestinal tract, and be non-toxic and non-pathogenic.

The DS109-B11 strain of the present invention may utilize glucose, mannitol, lactose, sucrose, maltose, salicin, xylose, arabinose, esculin, cellobiose, mannose, melezitose, raffinose, and sorbitol as carbon sources in terms of sugar utilization ability.

The DS108-B7 strain of the present invention may utilize glucose, mannitol, lactose, sucrose, maltose, salicin, xylose, arabinose, esculin, glycerol, cellobiose, mannose, melezitose, raffinose, sorbitol, rhamnose, and trehalose as carbon sources in terms of the sugar utilization ability.

In a specific example of the present invention, the sugar utilization ability of the two Bifidobacterium animalis spp. lactis strains was analyzed using the API 20A kit. As a result, it was confirmed that the DS109-B11 strain metabolizes glucose, mannitol, lactose, sucrose, maltose, salicin, xylose, arabinose, esculin, cellobiose, mannose, melezitose, raffinose, and sorbitol in terms of the sugar utilization ability, but may not metabolize gelatin, glycerol, rhamnose, and trehalose. In addition, it was confirmed that the DS108-B7 strain metabolizes glucose, mannitol, lactose, sucrose, maltose, salicin, xylose, arabinose, esculin, glycerol, cellobiose, mannose, melezitose, raffinose, sorbitol, rhamnose, and trehalose in terms of the sugar utilization ability, but may not metabolize gelatin.

In addition, the two Bifidobacterium animalis spp. lactis strains of the present invention may not produce toxic substances, and the toxic substances may be, for example, indole or urea.

Furthermore, the two Bifidobacterium animalis spp. lactis strains of the present invention may not produce β-glucuronidase. The term “β-glucuronidase” refers to an enzyme that promotes hydrolysis. The β-glucuronidase is an enzyme that hydrolyzes various alcohols, phenols, amines, and the like into compounds (glucuronides) containing glucuronic acid, and is present in many organisms, including bacteria, fungi, plants, and animals. For example, it is known that glucuronic acid is secreted in human sweat and is involved in the production of substances that cause glandular odor through the metabolism of bacteria that inhabit the skin. In particular, it has been reported to be associated with colon cancer.

In addition, the two Bifidobacterium animalis spp. lactis strains of the present invention may not exhibit hemolysis. Hemolysis refers to the strain's ability to destroy red blood cells. For the strain to be safely used as the probiotic, the strain should not have the hemolysis.

In a specific embodiment of the present invention, the two Bifidobacterium animalis spp. lactis strains of the present invention were analyzed for their indole, urea, and β-glucuronidase production ability. As a result, the strains of the present invention were found to lack the production of toxic substances such as indole and urea, and were unable to produce β-glucuronidase associated with colon cancer. Therefore, it was confirmed that the strains of the present invention are safe as the probiotics.

Furthermore, in a specific embodiment of the present invention, the two Bifidobacterium animalis spp. lactis strains of the present invention were cultured in blood media and analyzed for α, β, and γ hemolysis. As a result, the two Bifidobacterium animalis spp. lactis strains of the present invention did not exhibit hemolysis, confirming their safety as the probiotics.

In addition, the two Bifidobacterium animalis spp. lactis strains of the present invention may have acid-resistance. The acid-resistance refers to the property of the strain to survive in an acidic environment, such as an environment with a PH of 5 or lower, for example, an environment with a pH of 4 or lower, an environment with a pH of 3.5 or lower, and particularly an environment with a pH of 3 or lower, even after exposure to an acidic environment for at least 1 hour, for example, at least 1.5 hours, at least 2 hours, at least 2.5 hours, and particularly at least 3 hours.

In a specific embodiment of the present invention, even when the two Bifidobacterium animalis spp. lactis strains were cultured for 3 hours under pH 3.0 conditions, it was confirmed that the two Bifidobacterium animalis spp. lactis strains exhibit a survival rate of 138% or higher, were still viable, and have excellent acid resistance.

In addition, the two Bifidobacterium animalis spp. lactis strains of the present invention may have bile-resistance. The bile-resistance refers to the property of the strains being able to survive in a biliary environment containing bile distributed from the gallbladder, and may refer to the property of being able to survive in a biliary environment containing bile, such as oxgall or bile salts, at a concentration of 5% or less, for example, 4.5% or less, 4% or less, 3.5% or less, and particularly 3% or less, for 6 hours or more, for example, 7 hours or more, 8 hours or more, 9 hours or more, 10 hours or more, 11 hours or more, and particularly 12 hours or more.

In a specific embodiment of the present invention, even when the two Bifidobacterium animalis spp. lactis strains were cultured for 24 hours in conditions containing 3% (w/v) bile salts, it was confirmed that the two Bifidobacterium animalis spp. lactis strains exhibit a survival rate of over 58% and thus is still well surviving, and have excellent bile tolerance.

In addition, the two Bifidobacterium animalis spp. lactis strains of the present invention may have intestinal adhesion ability. The intestinal adhesion ability refers to the property of the strain to adhere to intestinal cells and not be detached. When the intestinal adhesion ability is excellent, the strain may remain in the intestine for a longer period of time, thereby prolonging its effects as the probiotics in improving the intestinal flora, promoting bowel movements, and promoting intestinal health. Specifically, the adhesion ability of the strain to the intestine may refer to that the strain adheres to intestinal cells after reaching the intestine, and thus after 12 hours or more, such as 15 hours or more, 18 hours or more, 21 hours or more, particularly 24 hours or more, in the intestinal environment, 1.0% or more, such as 1.1% or more, 1.15% or more, 1.5% or more, 2.0% or more, 2.5% or more, or 2.54% or more, of the initial number of viable bacteria remain adhered to the intestinal cells.

In a specific embodiment of the present invention, two Bifidobacterium animalis spp. lactis strains of the present invention were treated with the Caco-2 cell line and cultured using glucose as a carbon source. After the two Bifidobacterium animalis spp. lactis strains of the present invention was washed with PBS solution, the intestinal adhesion rate (%) of the strains was evaluated. As a result, the DS108-B7 strain exhibited an intestinal adhesion rate of approximately 2.54%, and the DS109-B11 strain exhibited an intestinal adhesion ability of approximately 1.14%, confirming that they have intestinal adhesion ability.

In addition, the two Bifidobacterium animalis spp. lactis strains may not exhibit antibiotic resistance or may have low antibiotic resistance. The resistance may be extrinsic resistance, which refers to resistance caused by the introduction of antibiotic resistance genes into other bacteria from an external source through mobile genetic elements such as plasmids or transposon. Therefore, the two Bifidobacterium animalis spp. lactis strains of the present invention, which do not exhibit extrinsic antibiotic resistance, do not horizontally transfer resistance genes to harmful bacteria present in the intestine, thereby eliminating the concern that harmful bacteria in the intestine may acquire extrinsic resistance and cause antibiotic resistance.

The antibiotics to which the two Bifidobacterium animalis spp. lactis strains of the present invention do not exhibit resistance are not particularly limited, as long as they are known in the art to which the present invention pertains. Examples of the antibiotics include penicillin antibiotics, tetracyclines antibiotics, macrolide antibiotics, sulfonamide antibiotics, amphenicol antibiotics, aminoglycoside antibiotics, and the like, and specifically, may include ampicillin, gentamicin, erythromycin, clindamycin, and tetracycline.

In a specific embodiment of the present invention, antibiotic resistance was tested for the two Bifidobacterium animalis spp. lactis strains using MTS™ (MIC Test Strip) (Liofilchem). As a result, it was confirmed that the DS108-B7 strain of the present invention has antibiotic susceptibility to ampicillin, vancomycin, erythromycin, clindamycin, and chloramphenicol below the standard value of EFSA GUARD, and the DS109-B11 strain of the present invention has antibiotic susceptibility to ampicillin, vancomycin, gentamicin, streptomycin, erythromycin, tetracycline, and chloramphenicol below the standard value of EFSA GUARD. In particular, in Europe, it has been impossible to commercialize lactic acid bacteria that may horizontally transfer antibiotic resistance genes to other bacteria for a long period of time, and in Korea, the Ministry of Food and Drug Safety recently inserted a clause on antibiotic resistance transfer into the criteria for judging microorganisms as food ingredients, so the two Bifidobacterium animalis spp. lactis strains of the present invention have low antibiotic resistance, and therefore, are recognized as safe as the probiotics and have an advantage in being commercialized.

Meanwhile, the two novel Bifidobacterium animalis spp. lactis strains of the present invention may promote AMPK protein activity.

The AMPK is an enzyme that plays a crucial role in maintaining cellular energy homeostasis. The AMPK is composed of three subunits of α, β, and γ and is a protein complex that is evolutionarily well-conserved from yeast to humans. Increasing the AMPK protein activity in muscle may increase mitochondrial biogenesis and increase muscle motor function.

Specifically, the two novel Bifidobacterium animalis spp. lactis strains of the present invention may have activity in promoting the AMPK protein activity in muscle cells or activity in promoting muscle cell differentiation.

In addition, the two novel Bifidobacterium animalis spp. lactis strains of the present invention may promote the expression of electron transport chain-related proteins.

The electron transport chain-related proteins may be involved in endurance, energy supply, or recovery, and may be involved in increasing energy production and supply efficiency, and thus, may be associated with muscle exercise performance enhancement. Examples of the electron transport chain-related proteins within muscle cells may include mitochondrial complex III, mitochondrial complex IV, ATP synthase c, etc. The mitochondria serve as powerhouses that enable muscle contraction and relaxation by generating oxygen and ATP through a series of electron transport or redox reactions of mitochondrial complexes I to V. Therefore, the two novel Bifidobacterium animalis spp. lactis strains of the present invention may have the function of enhancing muscle exercise performance by promoting the expression of the electron transport chain-related proteins such as mitochondrial complexes I to V.

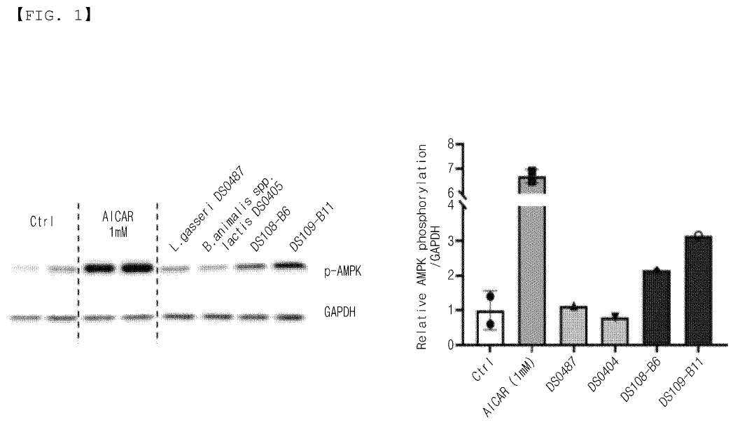

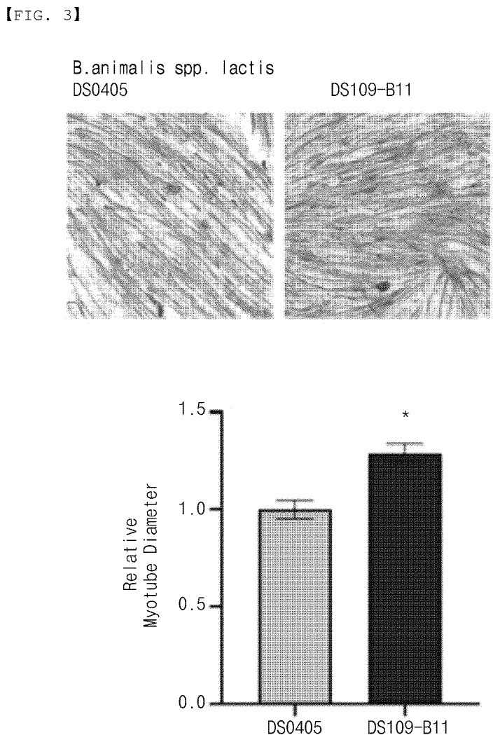

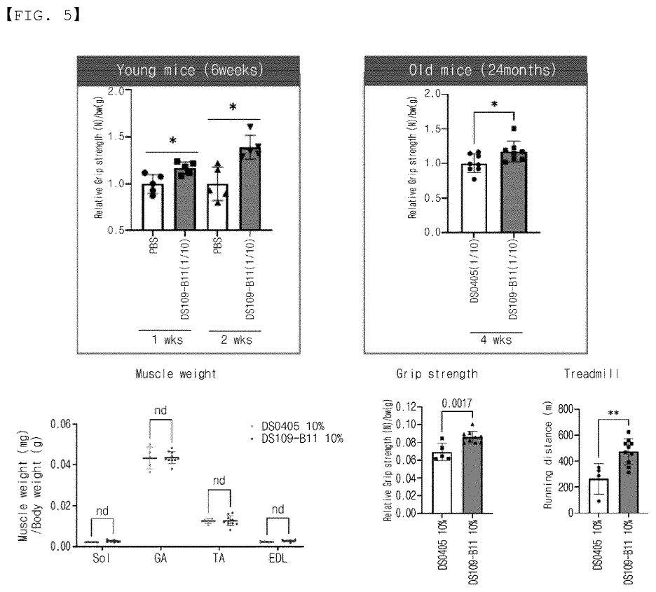

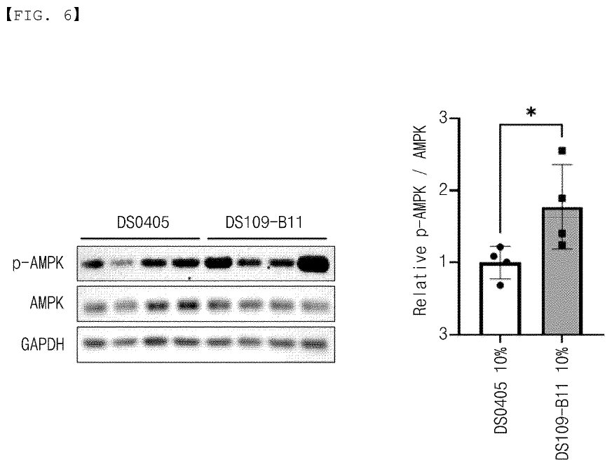

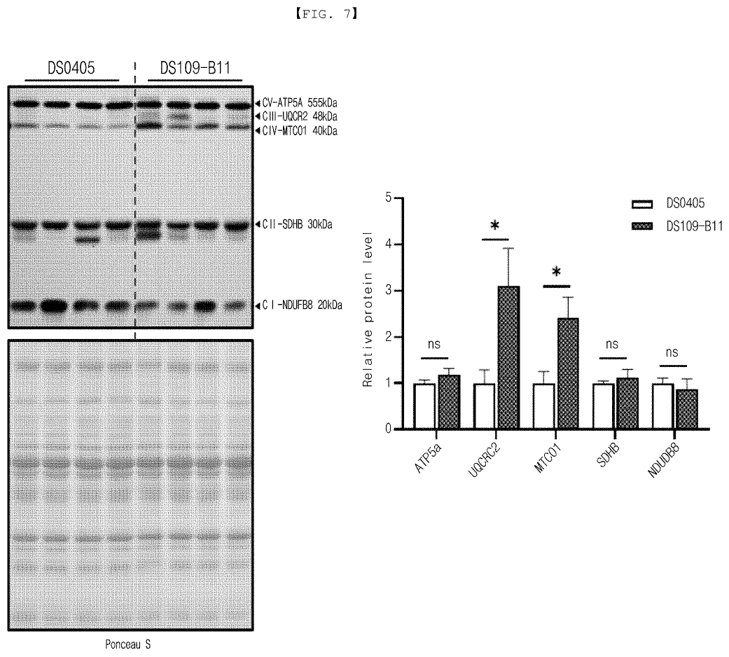

In a specific embodiment of the present invention, it was confirmed that, when the culture medium containing the novel Bifidobacterium animalis spp. lactis DS109-B11 strain of the present invention is applied to the mouse muscle cell line C2C12, which is widely studies used in on muscle differentiation and various intramuscular signaling mechanisms, the AMPK activity of the muscle cells was enhanced (see FIG. 1), and when treated with the two novel Bifidobacterium animalis spp. lactis DS109-B11 strains of the present invention, the muscle cell differentiation was increased (see FIG. 3), and the DS109-B11 strain improved the muscles of aged mice (see FIG. 5). In addition, it was confirmed that the AMPK activity (see FIG. 6) and the mitochondrial complex III and mitochondrial complex IV increased in aged mouse muscles treated with the DS109-B11 strain (see FIG. 7).

From the above results, the two novel Bifidobacterium animalis spp. lactis strains of the present invention are novel strains that promote the AMPK protein activity. Accordingly, the strains of the present invention may be effectively used for improving muscle function, enhancing exercise performance, or preventing, improving, or treating muscle diseases.

2. Novel Lactobacillus paracasei Strain

One aspect of the present invention provides a novel Lactobacillus paracasei strain.

The novel Lactobacillus paracasei strain of the present invention may comprise 16S rRNA having the base sequence of SEQ ID NO: 3, and in particular, may be the Lactobacillus paracasei DS108-B10 strain, deposited under accession No. KCTC 1534BP with the Korean Collection for Type Cultures (KCTC), on Mar. 10, 2023.

The novel Lactobacillus paracasei strain of the present invention may be a strain capable of inhabiting the intestines of humans or non-human animals, and may exert various health effects on humans or non-human animals while inhabiting the intestines. The novel Lactobacillus paracasei strain of the present invention may be a safe microorganism, as it does not exhibit toxicity or cause disease in humans or non-human animals, and may function as a microorganism that improves muscle function.

The DS108-B10 strain of the present invention may utilize glucose, mannitol, lactose, sucrose, maltose, salicin, xylose, arabinose, esculin, glycerol, cellobiose, mannose, melezitose, raffinose, sorbitol, rhamnose, and trehalose as carbon sources in terms of the sugar utilization ability.

In a specific example of the present invention, the sugar utilization ability of the Lactobacillus paracasei strain was analyzed using the API 20A kit. As a result, it was confirmed that the Lactobacillus paracasei strain is able to metabolize glucose, mannitol, lactose, sucrose, maltose, salicin, xylose, arabinose, esculin, glycerol, cellobiose, mannose, melezitose, raffinose, sorbitol, rhamnose, and trehalose in terms of the sugar utilization ability, but is unable to metabolize gelatin.

In addition, the Lactobacillus paracasei strain of the present invention may not produce toxic substances, and example of the toxic substances may include indole, urea, etc.

In addition, the Lactobacillus paracasei strain of the present invention may not exhibit hemolysis.

In a specific embodiment of the present invention, the production ability of the indole and urea of the Lactobacillus paracasei strain of the present invention was analyzed. As a result, the strain of the present invention was found to lack the ability to produce toxic substances such as indole and urea, confirming its safety as the probiotics.

In addition, in a specific embodiment of the present invention, the Lactobacillus paracasei strain of the present invention was cultured in blood medium and analyzed for α, β, and γ hemolysis. The Lactobacillus paracasei strain of the present invention did not exhibit hemolysis, confirming its safety as the probiotics.

In addition, the Lactobacillus paracasei strain of the present invention may possess intestinal adhesion ability. The intestinal adhesion ability refers to the property of the strain to adhere to intestinal cells and not be detached. When the intestinal adhesion ability is excellent, the strain may remain in the intestine for a longer period of time, thereby prolonging its effects as the probiotics in improving the intestinal flora, promoting bowel movements, and promoting intestinal health. Specifically, the adhesion ability of the strain to the intestine may refer to that the strain adheres to intestinal cells after reaching the intestine, and thus after 12 hours or more, such as 15 hours or more, 18 hours or more, 21 hours or more, particularly 24 hours or more, in the intestinal environment, 0.3% or more, such as 0.35% or more, 0.4% or more, 0.5% or more, 0.55% or more, 0.6% or more, or 0.66% or more, of the initial number of viable bacteria remain adhered to the intestinal cells.

In a specific embodiment of the present invention, the Lactobacillus paracasei strains of the present invention was treated with the Caco-2 cell line and cultured using the glucose as the carbon source. After the Lactobacillus paracasei strains of the present invention was washed with the PBS solution, the intestinal adhesion rate (%) of the strains was evaluated. As a result, the DS108-B10 strain exhibited the intestinal adhesion rate of approximately 0.66%, confirming its intestinal adhesion ability.

In addition, the Lactobacillus paracasei strain may not exhibit the antibiotic resistance or may have the low antibiotic resistance.

The description of resistance, antibiotics, etc., is identical to the description provided in section “1.” and will not be redundantly described.

The antibiotics to which the Lactobacillus paracasei strain of the present invention does not exhibit resistance are not particularly limited, as long as they are known in the art to which the present invention pertains. Examples of the antibiotics include penicillin antibiotics, tetracyclines antibiotics, macrolide antibiotics, sulfonamide antibiotics, amphenicol antibiotics, aminoglycoside antibiotics, and the like, and specifically, may include ampicillin, gentamicin, erythromycin, clindamycin, and tetracycline.

In a specific embodiment of the present invention, the antibiotic resistance was tested for the Lactobacillus paracasei strain using MTS™ (MIC Test Strip) (Liofilchem). As a result, the Lactobacillus paracasei strain of the present invention was confirmed to have antibiotic susceptibility below the EFSA GUARD standard for ampicillin, erythromycin, clindamycin, tetracycline, and chloramphenicol. In particular, in Europe, it has been impossible to commercialize lactic acid bacteria that may horizontally transfer antibiotic resistance genes to other bacteria for a long period of time, and in Korea, the Ministry of Food and Drug Safety recently inserted a clause on antibiotic resistance transfer into the criteria for judging microorganisms as food ingredients, so the Lactobacillus paracasei strain of the present invention has low antibiotic resistance, and therefore, is recognized as safe as the probiotics and have an advantage in being commercialized.

Meanwhile, the novel Lactobacillus paracasei strain of the present invention may promote the AMPK protein activity.

The AMPK is an enzyme that plays a crucial role in maintaining cellular energy homeostasis. The AMPK is composed of three subunits of α, β, and γ and is a protein complex that is evolutionarily well-conserved from yeast to humans. Increasing the AMPK protein activity in muscle may increase mitochondrial biogenesis and increase muscle motor function.

Specifically, the novel Lactobacillus paracasei strain of the present invention may have the activity in promoting the AMPK protein activity in the muscle cells or the activity in promoting the muscle cell differentiation.

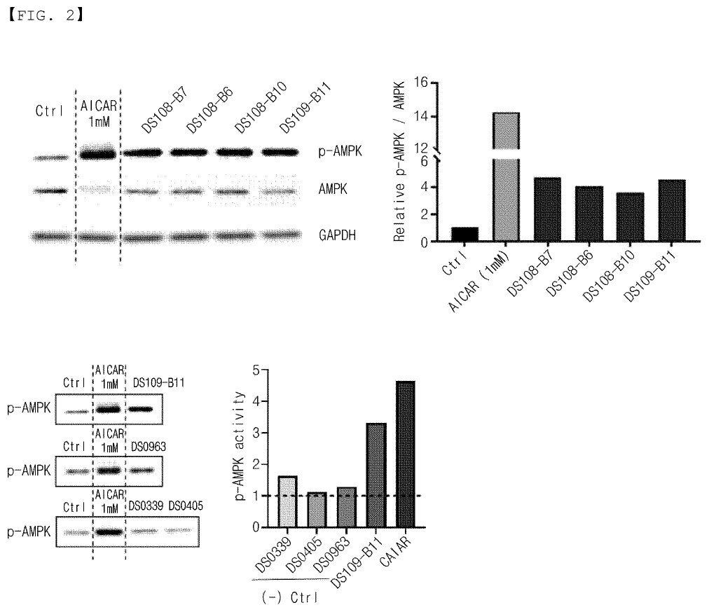

In a specific embodiment of the present invention, when the culture medium containing the novel Lactobacillus paracasei strain of the present invention was applied to the mouse muscle cells, the C2C12 that is the cell line widely used for muscle differentiation studying and various intramuscular signaling mechanisms, it was confirmed that the AMPK activity in the muscle cells was enhanced (see FIG. 2).

From the above results, the novel Lactobacillus paracasei strain of the present invention is a novel strain having the activity in promoting the AMPK protein activity, and accordingly, the strain of the present invention may be effectively used for improving muscle function or preventing, improving, or treating muscle diseases.

3. Novel Lactobacillus gasseri Strain

One aspect of the present invention provides a novel Lactobacillus gasseri strain.

The novel Lactobacillus gasseri strain of the present invention may comprise 16S rRNA having the base sequence of SEQ ID NO: 4, and in particular, may be the Lactobacillus gasseri DS108-B6 strain deposited under accession No. KCTC 15299BP with the Korean Collection for Type Cultures (KCTC), on Jan. 18, 2023.

The novel Lactobacillus gasseri strain of the present invention may be a strain capable of inhabiting the intestines of humans or non-human animals, and may exert various health effects on humans or non-human animals while inhabiting the intestines. The novel Lactobacillus gasseri strain of the present invention may be a safe microorganism, as it does not exhibit toxicity or cause disease in humans or non-human animals, and may function as a microorganism that improves muscle function.

The DS108-B6 strain of the present invention may utilize glucose, mannitol, lactose, sucrose, maltose, salicin, xylose, arabinose, esculin, cellobiose, mannose, melezitose, raffinose, sorbitol, rhamnose, and trehalose as the carbon sources in terms of the sugar utilization ability.

In a specific example of the present invention, the sugar utilization of the Lactobacillus gasseri strain was analyzed using the API 20A kit. As a result, it was confirmed that the Lactobacillus gasseri strain is able to metabolize glucose, mannitol, lactose, sucrose, maltose, salicin, xylose, arabinose, esculin, cellobiose, mannose, melezitose, raffinose, sorbitol, rhamnose, and trehalose in terms of the sugar utilization ability, but is unable to metabolize gelatin and glycerol.

In addition, the Lactobacillus gasseri strain of the present invention may not produce toxic substances, and example of the toxic substances may include indole, urea, etc.

In addition, the Lactobacillus gasseri strain of the present invention may not produce β-glucuronidase.

In addition, the Lactobacillus gasseri strain of the present invention may not exhibit hemolysis.

The descriptions regarding β-glucuronidase, hemolysis, etc., are identical to those described in “1. Two novel Bifidobacterium animalis spp. lactis strains” and will not redundantly be described.

In a specific embodiment of the present invention, the production ability of the indole, urea, and β-glucuronidase of the Lactobacillus gasseri strain of the present invention was analyzed. As a result, the strains of the present invention were found to lack the production of toxic substances such as indole and urea, and were unable to produce β-glucuronidase associated with colon cancer. Therefore, it was confirmed that the strains of the present invention are safe as the probiotics.

In a specific embodiment of the present invention, the production ability of the indole and urea of the Lactobacillus paracasei strain of the present invention was analyzed. As a result, the strain of the present invention was found to lack the ability to produce toxic substances such as indole and urea, confirming its safety as the probiotics.

In addition, in a specific embodiment of the present invention, the Lactobacillus gasseri strain of the present invention was cultured in blood medium and analyzed for α, β, and γ hemolysis. The Lactobacillus gasseri strain of the present invention did not exhibit hemolysis, confirming its safety as the probiotics.

In addition, the Lactobacillus gasseri strain of the present invention may possess intestinal adhesion ability. The intestinal adhesion ability refers to the property of the strain to adhere to intestinal cells and not be detached. When the intestinal adhesion ability is excellent, the strain may remain in the intestine for a longer period of time, thereby prolonging its effects as the probiotics in improving the intestinal flora, promoting bowel movements, and promoting intestinal health. Specifically, the adhesion ability of the strain to the intestine may refer to that the strain adheres to intestinal cells after reaching the intestine, and thus after 12 hours or more, such as 15 hours or more, 18 hours or more, 21 hours or more, particularly 24 hours or more, in the intestinal environment, 1.0% or more, such as 2.0% or more, 3.0% or more, 4.0% or more, 5.0% or more, 5.5% or more, or in particularly, 5.76% or more, of the initial number of viable bacteria remain adhered to the intestinal cells.

In a specific embodiment of the present invention, the Lactobacillus gasseri strain of the present invention was treated with the Caco-2 cell line and cultured using the glucose as the carbon source. After the Lactobacillus gasseri strain of the present invention was washed with the PBS solution, the intestinal adhesion rate (%) of the strains was evaluated. As a result, the DS108-B6 strain exhibited the intestinal adhesion rate of approximately 5.76%, confirming its intestinal adhesion ability.

In addition, the Lactobacillus gasseri strain may not exhibit the antibiotic resistance or may have the low antibiotic resistance.

The description of resistance, the antibiotic resistance, etc., is identical to the description provided in “1. Two novel Bifidobacterium animalis spp. lactis (Bifidobacterium animalis spp. lactis) strains” and will not redundantly be described.

The antibiotics to which the Lactobacillus gasseri strain of the present invention does not exhibit resistance are not particularly limited, as long as they are known in the art to which the present invention pertains. Examples of the antibiotics include penicillin antibiotics, tetracyclines antibiotics, macrolide antibiotics, sulfonamide antibiotics, amphenicol antibiotics, aminoglycoside antibiotics, and the like, and specifically, may include ampicillin, gentamicin, erythromycin, clindamycin, and tetracycline.

In a specific embodiment of the present invention, the antibiotic resistance was tested for the Lactobacillus gasseri strain using MTS™ (MIC Test Strip) (Liofilchem). As a result, the Lactobacillus gasseri strain of the present invention was confirmed to have antibiotic susceptibility below the EFSA GUARD standard for ampicillin, erythromycin, clindamycin, tetracycline, and chloramphenicol. In particular, in Europe, it has been impossible to commercialize lactic acid bacteria that may horizontally transfer antibiotic resistance genes to other bacteria for a long period of time, and in Korea, the Ministry of Food and Drug Safety recently inserted a clause on antibiotic resistance transfer into the criteria for judging microorganisms as food ingredients, so the Lactobacillus gasseri strain of the present invention has low antibiotic resistance, and therefore, is recognized as safe as the probiotics and have an advantage in being commercialized.

Meanwhile, the novel Lactobacillus gasseri strain of the present invention may promote the AMPK protein activity.

The AMPK is an enzyme that plays a crucial role in maintaining cellular energy homeostasis. The AMPK is composed of three subunits of α, β, and γ and is a protein complex that is evolutionarily well-conserved from yeast to humans. Increasing the AMPK protein activity in muscle may increase mitochondrial biogenesis and increase muscle motor function.

Specifically, the novel Lactobacillus gasseri strain of the present invention may have the activity in promoting the AMPK protein activity in the muscle cells or the activity in promoting the muscle cell differentiation.

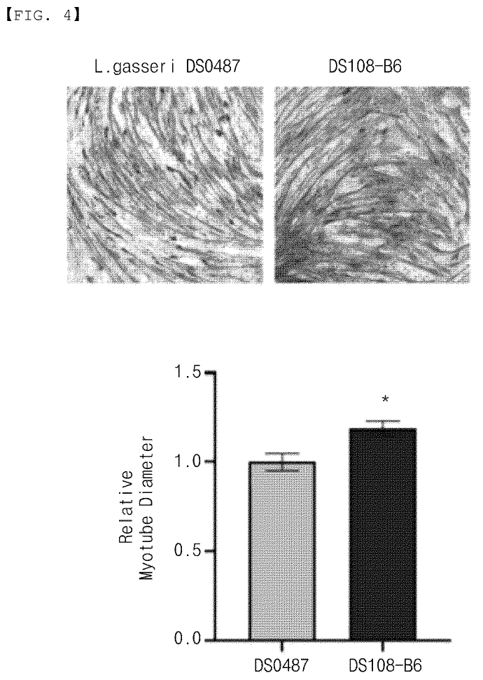

In a specific embodiment of the present invention, when the culture medium containing the novel Lactobacillus gasseri strain of the present invention was applied to mouse muscle cells, C2C12 that is a cell line widely used for studying muscle differentiation and various intramuscular signaling mechanisms, it was confirmed that not only did it enhance the AMPK activity in the muscle cells (see FIG. 1) but also increased muscle cell differentiation (see FIG. 4).

From the above results, the novel Lactobacillus gasseri strain of the present invention is a novel strain having the activity in promoting the AMPK protein activity, and accordingly, the strain of the present invention may be effectively used for improving muscle function or preventing, improving, or treating muscle diseases.

### 4. Composition Comprising Strain of the Present Invention

Another aspect of the present invention provides a composition comprising, as an active ingredient, at least one selected from the group consisting of the novel Bifidobacterium animalis spp. lactis strains, Lactobacillus paracasei strain, Lactobacillus gasseri strain, or their combinations, a culture medium of the strain or their combinations, a concentrate of the culture medium, a dried product of the culture medium, and an extract of the culture medium.

The culture medium is obtained by culturing a strain selected from the novel Bifidobacterium animalis spp. lactis strains, Lactobacillus paracasei strain, Lactobacillus gasseri strain, and their combinations of the present invention. The culture medium may be the culture medium itself containing cells of the strain, or a culture supernatant obtained by removing cells therefrom, or may be a filtrate, concentrate, or dried product thereof. The culture medium from which cells have been removed may contain components produced and secreted by the novel strain of the present invention, for example, metabolites.

The concentrate increases the solid concentration of the culture medium, and may be a concentrate of a culture medium containing cells of a strain selected from the novel Bifidobacterium animalis spp. lactis strains, Lactobacillus paracasei strain, Lactobacillus gasseri strain, and their combinations thereof of the present invention, or a concentrate of a culture supernatant from which cells of the novel Bifidobacterium animalis spp. lactis strain of the present invention have been removed. The concentrate may be concentrated by vacuum concentration, plate concentration, thin-film concentration, etc., but is not limited thereto, and may be performed, for example, at a temperature of 40 to 60° C. using the known concentrator. The content of the culture medium included in the composition of the present invention may be appropriately adjusted depending on the concentration of the concentrate.

The dried product includes, but is not limited to, a dried product dried by a method such as freeze drying, vacuum drying, hot air drying, spray drying, reduced pressure drying, spray drying, foam drying, high-frequency drying, or infrared drying.

The extract refers to an extract extracted from the culture medium or its concentrate, and may include an extract, a dilution or concentrate of the extract, a dried product obtained by drying the extract, a conditioned or purified product thereof, or fractions thereof.

In addition, the composition is suitable for ingestion with a strain selected from the novel Bifidobacterium animalis spp. lactis strains, a Lactobacillus paracasei strain, a Lactobacillus gasseri strain, and their combinations of the present invention, and may additionally include other known ingredients or lactic acid bacteria that enhance muscle cell differentiation upon ingestion.

The composition comprising the novel Bifidobacterium animalis spp. lactis strains, Lactobacillus paracasei strain, Lactobacillus gasseri strain, and their combinations of the present invention may be formulated using a carrier, an excipient, and/or an additive according to a method readily practiced by a person skilled in the art to which the present invention pertains, and may be manufactured in a unit-dose form or manufactured by being filled into in a multi-dose container. In this case, the formulation may be in the form of a solution, a suspension, or an emulsion in an oil or aqueous medium, or in the form of an extract, powder, granule, tablet, capsule, gel (e.g., hydrogel), or lyophilizer, and may additionally include a dispersant, a stabilizer, or a cryoprotectant as additives.

Specifically, the lyophilizer includes lyophilizing a strain together with a cryoprotectant and using the strain in powder form, and may be skimmed milk powder, maltodextrin, dextrin, trehalose, maltose, lactose, mannitol, cyclodextrin, glycerol, and/or honey. In addition, the formulation may be mixed with a preservative, adsorbed, dried, and solidified for use. The preservative may be diatomaceous earth, activated carbon, and/or defatted steel.

The composition comprising a strain selected from the novel Bifidobacterium animalis spp. lactis strains, Lactobacillus paracasei strain, Lactobacillus gasseri strain, and their combinations of the present invention may be prepared through a step of mixing at least one selected from the group consisting of a strain, a culture medium of the strain, a concentrate of the culture medium, a dried product of the culture medium, and an extract of the culture medium with any one of a carrier, an excipient, or an additive.

The strain, carrier, excipient, and additive are as described above. When the cryoprotectant is used as the additive, the novel strain of the present invention and the cryoprotectant may be mixed, the mixture may be frozen at −45° C. to −30° C., dried at 30° C. to 40° C., and ground in a mixer to produce a lyophilized powder. Specifically, the freezing process may be vacuum freezing at a temperature of −45° C. to −30° C. and a pressure of 5 to 50 mTorr for 65 to 75 hours.

When the composition of the present invention further includes a cryoprotectant, damage or death of the strain may s of the be suppressed during the lyophilizing process composition. The lyophilized composition offers advantages during storage, distribution, and preservation. In addition, the lyophilized composition may be ingested in powder form. In this case, the strain in the composition may exhibit its activity as it grows or metabolizes in the body.

### 5. Use of Novel Strain of the Present Invention or Composition Comprising the Same

Another aspect of the present invention provides a use of a composition comprising a strain selected from the novel Bifidobacterium animalis spp. lactis strains, Lactobacillus paracasei strain, Lactobacillus gasseri strain, and their combinations, for improving muscle function, enhancing muscle exercise performance, or preventing or treating muscle disease.

The improvement in muscle function may refer to muscle exercise performance enhancement, and specifically, the muscle exercise performance enhancement may include muscle fatigue recovery, endurance enhancement, and neuromuscular activation. In particular, the composition may be a food, feed, or pharmaceutical. When the composition is a food, it may be a health functional food composition for improving muscle function or a general food composition. If the composition is a feed, it may be a feed composition for improving muscle function or a feed additive composition. When the composition is a pharmaceutical, it may be a pharmaceutical composition for preventing or treating muscle diseases.

In one implementation example of the present invention, when the composition is a health functional food composition for improving muscle function or a general food composition, the health functional food composition or food composition may enhance the AMPK activity in muscle cells, increase a surface area of muscle, increase muscle differentiation, or increase mitochondrial synthesis.

When the health functional food composition of the present invention is used as a food additive, it may be added directly to a food or used in combination with other foods or food ingredients, and may be used appropriately according to conventional methods. The amount of the active ingredient may be appropriately used depending on its intended use (prevention or improvement). Generally, when manufacturing a food or beverage, the health functional food composition of the present invention is added in an amount of 15 parts by weight or less, preferably 10 parts by weight or less, relative to the raw material. However, for long-term consumption for health purposes, the amount may be less than or equal to the above range. Since there are no safety issues, the active ingredient may be used in an amount greater than or equal to the above range.

There are no specific restrictions on the type of health functional food or food. Examples of the health functional food or food include meat, sausage, bread, chocolate, candy, snacks, confectionery, pizza, ramen, other noodles, gum, dairy products including ice cream, various soups, beverages, tea drinks, alcoholic beverages, vitamin complexes, dairy products, fermented milk, etc., and encompass all health functional foods or foods in the common sense.

In particular, the health functional food described above is a highly effective medical food processed to efficiently exhibit bioregulatory functions in addition to providing nutrition, and may provide beneficial health benefits, such as regulating nutrients for the structure and function of the human body or promoting physiological functions. The health functional food described above may be manufactured using methods commonly used in the technical field of the present invention, and may be manufactured by adding raw materials and ingredients commonly added in the art. In addition, the health functional food may be manufactured in a variety of forms without limitation, as long as it is a formulation recognized as a health functional food. Unlike general drugs, the health functional food has the advantage of being free of side effects that may occur with long-term use, as it uses food as a raw material, and is highly portable.

The health functional food of the present invention includes ingredients commonly added during food manufacturing, such as proteins, carbohydrates, fats, nutrients, and seasonings. For example, when manufactured as a drink, the health functional food may include natural carbohydrates or flavorings as additional ingredients in addition to the active ingredient. The natural carbohydrate is preferably a monosaccharide (e.g., glucose, fructose, etc.), a disaccharide (e.g., maltose, sucrose, etc.), an oligosaccharide, polysaccharide (e.g., dextrin, cyclodextrin, etc.), or a sugar alcohol (e.g., xylitol, sorbitol, erythritol, etc.). The flavoring agent may be a natural flavoring agent (e.g., thaumatin, stevia extract, etc.) or a synthetic flavoring agent (e.g., saccharin, aspartame, etc.).

In addition to the health functional food composition, the composition may further contain various nutrients, vitamins, electrolytes, flavoring agents, coloring agents, pectic acid and its salts, alginic acid and its salts, organic acids, protective colloid thickeners, pH adjusters, stabilizers, preservatives, glycerin, alcohol, carbonating agents used in carbonated beverages, etc. The ratio of these added ingredients is not particularly critical, but is typically selected within the range of 0.01 to 0.1 parts by weight per 100 parts by weight of the health functional food composition of the present invention.

In addition, in another embodiment of the present invention, if the composition is a feed composition or feed additive composition for improving muscle function, the feed composition or feed additive composition may be used in the diet of animals for the purpose of improving muscle function. The feed additive of the present invention corresponds to a supplementary feed under the Feed Management Act.

The term “feed” in the present invention may refer to any natural or artificial diet, meal, etc., or the components of such meal, intended for or suitable for eating, ingesting, and digesting by animals. The type of feed is not particularly limited, and any feed commonly used in the art may be used. Non-limiting examples of the feed include plant-based feeds such as grains, roots, fruits, food processing by-products, algae, fibers, pharmaceutical by-products, oils, starches, meal, or grain by-products; Examples of animal feed include proteins, inorganic substances, fats, minerals, oils, single-cell proteins, zooplankton, and food. These may be used alone or in combination of two or more.

In another embodiment of the present invention, when the composition is a pharmaceutical composition for preventing or treating muscle diseases, the muscle disease may be a condition in which muscle development is insufficient, muscle motor function is reduced, or muscle mass is reduced. This includes the muscle condition of fetuses, newborns, infants, and children still in the developmental or growth period, as well as aged muscle conditions. In addition, the muscle disease may be a condition in which muscle development is insufficient compared to the average level of muscle development during the same period of development or growth. The muscle disease encompasses all diseases caused by or related to insufficient muscle development. In addition, the muscle disease encompasses all diseases caused by or related to insufficient muscle motor function. In addition, the aforementioned muscle disease may be a condition in which muscle mass is reduced due to various causes, such as aging, genetic factors, or external environmental factors, and encompasses all diseases caused by or related to such muscle mass reduction. Specifically, the muscle disease may include sarcopenia or the resulting movement disorders or gait disorders, but is not limited thereto.

In addition, the prevention may refer to any action that blocks, suppresses, or delays symptoms caused by underdeveloped muscle, impaired motor function, or reduced muscle mass. In addition, the treatment may refer to any action that improves or benefits symptoms caused by muscle development disorders or reduction.

Meanwhile, the pharmaceutical composition may be formulated in unit dose form or by being filled into a multi-dose container using pharmaceutically acceptable carriers and/or excipients, according to methods readily accessible to those skilled in the art. In this case, the formulation may be a solution, suspension, or emulsion in an oil or aqueous medium, or an extract, powder, granules, tablets, capsules, or gel (e.g., hydrogel), and may additionally include a dispersant or stabilizer.

In addition, the strain contained in the pharmaceutical composition may be delivered in a colloidal pharmaceutically acceptable carrier, such as a suspension, powder, saline solution, lipids, liposomes, microspheres, or nano-spherical particles. These may form composites or may be associated with a carrier vehicle and may be delivered in vivo using carrier systems known in the art, such as lipids, liposomes, microparticles, gold, nanoparticles, polymers, condensation agents, polysaccharides, polyamino acids, dendrimers, saponins, adsorption enhancing substances, or fatty acids.

In addition, pharmaceutically acceptable carriers may include, but are not limited to, lactose, dextrose, sucrose, sorbitol, mannitol, starch, acacia, gum, calcium phosphate, alginate, gelatin, calcium silicate, microcrystalline cellulose, polyvinyl pyrrolidone, cellulose, water, syrup, methyl cellulose, methyl hydroxybenzoate, propyl hydroxybenzoate, talc, magnesium stearate, and mineral oil, which are commonly used in formulations. In addition to the above ingredients, lubricants, wetting agents, sweeteners, flavoring agents, emulsifiers, suspending agents, preservatives, and the like may be additionally included. Suitable pharmaceutically acceptable carriers and formulations are described in detail in Remington's Pharmaceutical Sciences (19th ed., 1995).

The pharmaceutical composition according to the present invention may be administered orally or parenterally during clinical administration and may be used in the form of general pharmaceutical formulations. That is, the pharmaceutical composition of the present invention may be administered in various oral and parenteral dosage forms during actual clinical administration. When formulated, it is prepared using diluents or excipients such as commonly used fillers, bulking agents, binders, wetting agents, disintegrants, and surfactants. Solid preparations for oral administration include tablets, pills, powders, granules, capsules, etc. These solid preparations are prepared by mixing herbal extracts or fermented herbal products with at least one excipient, such as starch, calcium carbonate, sucrose or lactose, or gelatin. In addition to simple excipients, lubricants such as magnesium stearate talc are also used. Liquid preparations for oral administration include suspensions, oral solutions, emulsions, and syrups. In addition to commonly used simple diluents such as water and liquid paraffin, they may contain various excipients such as wetting agents, sweeteners, flavoring agents, and preservatives. Preparations for parenteral administration include sterile aqueous solutions, non-aqueous solvents, suspensions, emulsions, lyophilized preparations, and suppositories. Non-aqueous solvents and suspensions may include propylene glycol, polyethylene glycol, vegetable oils such as olive oil, and injectable esters such as ethyl oleate, etc. Suppository bases include Witepsol, Macrogol, Tween 61, cacao butter, laurin butter, glycerol, gelatin, etc.

The pharmaceutical composition the of present invention may be used alone or in combination with methods using surgery, radiotherapy, hormone therapy, chemotherapy, and biological response modifiers for the prevention and treatment of muscle diseases.

The concentration of the active ingredient in the composition of the present invention may be determined based on therapeutic purpose, the patient's condition, the required duration, etc., and is not limited to a specific concentration range. The pharmaceutical composition of the present invention is administered in a pharmaceutically effective amount. In the present invention, a “pharmaceutically effective amount” means an amount sufficient to treat a disease at a reasonable benefit/risk ratio applicable to medical treatment. The effective dosage level may be determined based on the type and severity of the patient's disease, the activity and sensitivity of the drug, the time of administration, the route of administration, and the excretion rate, the treatment period, concurrent medications, and other factors well known in the medical field. The pharmaceutical composition of the present invention may be administered as a separate therapeutic agent or in combination with other therapeutic agents for improving muscle aging, and may be administered simultaneously, separately, or sequentially with conventional therapeutic agents, and may be administered singly or in multiple doses. Considering all of the above factors, it is important to administer an amount that achieves maximum effect with the minimum amount possible without adverse effects, and this may be readily determined by those skilled in the art.

Specifically, the effective dose of the pharmaceutical composition of the present invention may vary depending on the patient's age, sex, condition, and weight, the absorption rate, inactivation rate, and excretion rate of the active ingredient in the body, the type of disease, and concomitant medications. The effective dose may also vary depending on the route of administration, muscle mass or the severity of the muscle disease, sex, weight, age, etc.

Another aspect of the present invention provides a method for preventing or treating muscle disease, comprising administering the pharmaceutical composition to a subject.

The subject may be a human or non-human animal, and may be a subject whose muscles are less developed than those of a fully developed human or non-human animal, or may be in the developmental or growth phase. In addition, the subject may be a human or non-human animal whose muscles are less developed than the average level of muscle development during the same developmental or growth phase during which muscle development is in progress. In addition, the subject may be a subject with reduced muscle motor function. In addition, the subject may be a human or non-human animal whose muscle motor function is less than the average level of muscle motor function during the same time period. In addition, the subject may be a subject whose muscles have decreased due to various causes, such as aging. In addition, the subject may be a human or non-human animal whose muscle mass is less than the average level during adolescence, middle age, or middle-to-late-adulthood.

Descriptions regarding the formulation, administration method, dosage, and concentration of the active ingredient contained in the pharmaceutical composition are the same as those described above.

Hereinafter, examples of the present invention will be described in detail.

However, the following examples specifically illustrate the present invention, and the contents of the present invention are not limited by the following examples.

### Example 1

### Establishment of Novel Strains Present Invention

### 1-1. Isolation and Identification of Novel Strains of the Present Invention

The DS109-B11, DS108-B10, DS108-B7, and DS108-B6 strains were isolated from human feces. The 16S rRNA base sequences of the DS109-B11, DS108-B10, DS108-B7, and DS108-B6 strains were analyzed by PCR using the universal primers 27F (5′-AGAGTTTGATCMTGGCTCA-3′: SEQ ID NO: 5) and 1492R (5 ‘-TACGGYTACCTTGTTACGACTT-3’: SEQ ID NO: 6).

As a result, the 16S rRNA base sequence of the DS109-B11 strain was as shown in SEQ ID NO: 1, which was confirmed to have 99.93% homology with the previously reported Bifidobacterium animalis spp. lactis standard strain. The 16S rRNA base sequence of the DS108-B7 strain was as shown in SEQ ID NO: 2, which was confirmed to have 100% homology with the previously reported Bifidobacterium animalis spp. lactis standard strain. The 16S rRNA base sequence of the DS108-B10 strain was as shown in SEQ ID NO: 3, which was confirmed to have 99.93% the homology with previously reported Lactobacillus paracasei standard strain. The 16S rRNA base sequence of the DS108-B6 strain was identified as SEQ ID NO: 4, demonstrating 99.73% homology with the previously reported Lactobacillus gasseri standard strain.

Sequence comparison with the standard strain was performed on the EZ BioCloud server (https://eabiocloud.net/identify).

Therefore, the DS109-B11, DS108-B10, DS108-B6, and DS108-B7 strains were designated as shown in Table 1 below and deposited with the Korean Collection for Type Cultures (KCTC) of the Korea Research Institute of Bioscience and Biotechnology on Jan. 18, 2023, and Mar. 10, 2023, respectively, and assigned accession numbers as shown in Table 1.

TABLE 1

| Strain | Name | Accession No. |
|---|---|---|
| DS109- | Bifidobacterium animalis spp lactis | KCTC |
| B11 | DS109-B11 | 15297BP |
| DS108- | Bifidobacterium animalis spp lactis | KCTC |
| B7 | DS108-B7 | 15298BP |
| DS108- | Lactobacillus paracasei DS108-B10 | KCTC |
| B10 | | 15343BP |
| DS108- | Lactobacillus gasseri DS108-B6 | KCTC |
| B6 | | 15299BP |

The four novel strains described above were inoculated onto MRS (Man, Rogosa, and Sharpe) medium and cultured under anaerobic conditions at 37° C. for 24 to 36 hours to reach stationary phase. Next, the culture medium was centrifuged at 3,000× g for 10 minutes, and the supernatant was collected. The supernatant was then sterilized at 65° C. for 30 minutes, passed through a 0.22 μm filter, and stored at −80° C. for use in the experiments.

### 1-2. Whole-Genome Analysis of Novel Strains of the Present Invention

The whole-genome of the novel strains of the present invention isolated as described above were analyzed. Specifically, complete whole genome sequencing (WGS) and draft genome analysis were performed, and the identity and functionality of the strains were investigated, including genome homology analysis with the standard strain, phylogenomic characterization, and analysis of specific genes and metabolic pathways, using the genome analysis results. In addition, to investigate genome-level stability, the complete genome was secured, the amino acid sequence of the coding sequence (CDS) was extracted, and then the homology of toxic genes was searched from the predicted protein-coding genes in the genome to confirm the presence of toxic genes. The genome homology analysis was searched using the Antibiotic Resistance Genes DB and the Comprehensive Antibiotic Resistance DB. In addition, in order to search 1 for functional genes of the strains of the present invention, errors in the sequencing results were corrected through manual curation of the assembled genome, structural prediction of the genes and functional gene tagging were performed, and a genome map was created. Specifically, a standard strain and a functional comparison strain were selected from the GeneBank database, CDSs were extracted from the genome sequences, and orthologous gene clustering of the predicted protein sequences was performed using A11 to A11 BLAST and the Markov Cluster Algorithm (MCL). The genetic characteristics of the strains of the present invention were then analyzed by comparing and analyzing the genes of the comparison strains with those of the strains of the present invention.

As a result, as shown in Table 2, the DS109-B11 strain of the present invention had a whole-genome of 1.93 Mb, the DS108-B7 strain of the present invention had a whole-genome of 1.91 Mb, the DS108-B10 strain of the present invention had a whole-genome of 3.07 Mb, and the DS108-B6 strain of the present invention had a whole-genome of 2.10 Mb. In addition, it was confirmed that the two Bifidobacterium animalis spp. lactis strains and the Lactobacillus gasseri strain of the present invention do not possess resistance or toxicity genes, and thus it was found that they may be safely utilized as the probiotics.

TABLE 2

| | | | Whole- | |
|---|---|---|---|---|
| | | | genome | Resistance and |
| | Strain name | Isolate | (Mb) | toxicity genes |
| 1 | Bifidobacterium | Feces | 1.93 | — |
| | animalis spp lactis | | | |
| | DS109-B11 | | | |
| 2 | Bifidobacterium | Feces | 1.91 | — |
| | animalis spp lactis | | | |
| | DS108-B7 | | | |
| 3 | Lactobacillus | Feces | 3.07 | efaA (61%) * |
| | paracasei DS108-B10 | | | |
| 4 | Lactobacillus | Feces | 2.10 | — |
| | gasseri DS108-B6 | | | |

### Example 2

Confirmation of characterization of Novel Strains

To determine whether the four novel strains isolated and identified in Example 1-1 possessed properties suitable for use as the probiotics, tests were conducted on MRS medium for their sugar utilization, acid tolerance, bile tolerance, intestinal adhesion ability, and the antibiotic resistance.

### 2-1. Verification of Sugar Utilization, Toxin β-Glucuronidase Productivity, Gelatin Degradation, Productivity, and Hemolysis

To determine the sugar utilization, toxicant production, gelatin degradation, β-glucuronidase production, and hemolysis of the four strains of the present invention, the novel strains of the present invention were spread on MRS agar. The sugar utilization, toxicant production, and gelatin degradation assays were conducted using the API 20A kit (Bio-Merieux, France). Following the instructions provided, strain colonies were suspended in API 20A medium to a McFarland turbidity of 3 or higher and then inoculated into each well. In addition, since the β-glucuronidase has been reported to be associated with colon cancer, the productivity of β-glucuronidase was analyzed using the API ZYM kit. Next, the hemolytic activity was assessed by confirming α, β, and γ hemolysis on sheep blood agar plates.

The sugars identified in this experiment were glucose (GLU), mannitol (MAN), lactose (LAC), sucrose (SAC), maltose (MAL), salicin (SAL), xylose (XYL), arabinose (ARA), gelatin (GEL), esculin (ESC), glycerol (GLY), cellobiose (CEL), mannose (MNE), melezitose (MLZ), raffinose (RAF), sorbitol (SOR), rhamnose (RHA), and trehalose (TRE). The toxic substances identified in this experiment were indole and urea.

The prepared bacterial suspensions of each strain were inoculated into sugar tubes and cultured for 24 hours. A drop of BCP reagent was added to each sugar tube (except URE, GEL, and ESC), and the resulting reaction results were read according to the reading table. For each reaction, a positive result was indicated as +, and a negative result was indicated as −. The results are presented in Table 3.

TABLE 3

| | L. paracasei | L. gasseri | B. animalis | B. animalis |
|---|---|---|---|---|
| | DS108-B10 | DS108-B6 | DS108-B7 | DS109-B11 |
| D-glucose | + | + | + | + |
| D-mannitol | + | + | + | + |
| D-Lactose | + | + | + | + |
| sucrose | + | + | + | + |
| D-maltose | + | + | + | + |
| salicin | + | + | + | + |
| D-xylose | + | + | + | + |
| L-arabinose | + | + | + | + |
| gelatin | − | − | − | − |
| esculin | + | + | + | + |
| glycerol | + | − | + | − |
| D-cellobiose | + | + | + | + |
| D-mannose | + | + | + | + |
| D-melezitose | + | + | + | + |
| D-raffinose | + | + | + | + |
| D-sorbitol | + | + | + | + |
| D-rhamnose | + | + | + | − |
| D-trehalose | + | + | + | − |
| Indol | − | − | − | − |
| production | | | | |
| Urea | − | − | − | − |
| production | | | | |
| Gelatin | − | − | − | − |
| Degradability | | | | |
| β- | + | − | − | − |
| glucuronidase | | | | |
| Hemolysis | − | − | − | − |

As a result, as described in Table 3 above, it was confirmed that the novel DS108-B10 and DS108-B7 strains of the present invention metabolize glucose, mannitol, lactose, sucrose, maltose, salicin, xylose, arabinose, esculin, glycerol, cellobiose, mannose, melezitose, raffinose, sorbitol, rhamnose, and trehalose. It was confirmed that the novel DS108-B6 strain of the present invention metabolizes glucose, mannitol, lactose, sucrose, maltose, salicin, xylose, arabinose, esculin, cellobiose, mannose, melezitose, raffinose, sorbitol, rhamnose, and trehalose. The novel DS109-B11 strain of the present invention was confirmed to metabolize glucose, mannitol, lactose, sucrose, maltose, salicin, xylose, arabinose, esculin, cellobiose, mannose, melezitose, raffinose, and sorbitol. In addition, the four strains of the present invention were confirmed not to produce toxic substances such as indole and urea, not to degrade gelatin, and not to exhibit hemolysis. Meanwhile, the DS108-B6, DS108-B7, and DS109-B11 strains of the present invention were found not to produce β-glucuronidase.

### 2-2. Confirmation of Acid Resistance

The four strains of the present invention isolated and identified in Example 1 were inoculated into MRS medium and pre-cultured at 37° C. The concentration of each strain was adjusted to an OD₆₀₀ of 1. Then, each strain was inoculated at a concentration of 1% (v/v) into 10 mL of MRS medium adjusted to pH 3.0, cultured for 3 hours, and the survival rate was confirmed.

TABLE 4

| | Total | Total | |
|---|---|---|---|
| | bacterial | bacterial | Survival |
| | count of 0 h | count of 3 h | rate |
| Strain | (CFU) | (CFU) | (%) |
| Bifidobacterium | 3.4 × 10⁵ | 4.7 × 10⁵ | 138 |
| animalis spp lactis | | | |
| DS109-B11 | | | |
| Bifidobacterium | 2.5 × 10⁶ | 3.5 × 10⁶ | 140 |
| animalis spp lactis | | | |
| DS108-B7 | | | |
| Lactobacillus | 5.9 × 10⁶ | 2.5 × 10⁵ | 4.2 |
| gasseri DS108-B6 | | | |

As a result, as described in Table 4 above, both novel Bifidobacterium animalis spp. lactis strains of the present invention exhibited a survival rate of over 100%, confirming their basic resistance to acidic environments.

### 2-3. Confirmation of Bile Resistance

The four strains of the present invention were inoculated into 10 ml of MRS medium containing 3% (w/v) bile salts (Oxoid™) at a concentration of 10⁶ CFU/mL, cultured for 12 hours, and then the survival rate was confirmed.

TABLE 5

| | Total | Total | |
|---|---|---|---|
| | bacterial | bacterial | Survival |
| | count of 0 h | count of 12 h | rate |
| Strain | (CFU) | (CFU) | (%) |
| Bifidobacterium | 5.0 × 10⁵ | 3.0 × 10⁵ | 60.0 |
| animalis spp lactis | | | |
| DS109-B11 | | | |
| Bifidobacterium | 2.9 × 10⁵ | 1.7 × 10⁶ | 58.0 |
| animalis spp lactis | | | |
| DS108-B7 | | | |
| Lactobacillus | 2.9 × 10⁵ | 0 | 0 |
| gasseri DS108-B6 | | | |

NC = Not countable.

As a result, as described in Table 5 above, the two novel Bifidobacterium animalis spp. lactis strains of the present invention exhibited a survival rate of over 58% in an environment containing bile, confirming their bile tolerance.

### 2-4. Confirmation of Antibiotic Resistance

To confirm the antibiotic resistance of the four strains of the present invention, two Bifidobacterium animalis spp. lactis strains were prepared by spreading them on MRS agar medium. Antibiotic resistance tests were conducted using MTS™ (MIC Test Strip) (Liofilchem) according to the manufacturer's instructions for antibiotics commonly used in EFSA (European Food Safety Authority) guidelines.

The antibiotics used in this experiment were ampicillin (Amp), vancomycin (Van), gentamicin (Gen), kanamycin (Kan), streptomycin (Str), erythromycin (Ery), clindamycin (Cln), tetracycline (Tet), and chloramphenicol (Chr). The minimum inhibitory concentration (MIC) was defined as the concentration at which the antibiotic strip intersected the inhibition zone.

The four strains of the present invention were spread on the prepared medium, antibiotic strips were placed, and the cells were incubated at 37° C. for 48 hours. The minimum inhibitory concentration (MIC) at which growth was inhibited was measured. The results are presented in Table 6.

TABLE 6

| EFSA Standard | Amp | Van | Gen | Kan | Str | Ery | Cln | Tet | Chr |
|---|---|---|---|---|---|---|---|---|---|
| Bifidobacterium | 2 | 2 | 64 | n.r | 128 | 1 | 1 | 8 | 4 |
| B. animalis DS108-B7 | 0.09 | 0.5 | >258 | — | 192 | 0.12 | 0.03 | 12 | 2 |
| B. animalis DS109-B11 | 0.5 | 1 | 64 | — | 32 | 1 | 8 | 1.5 | 2 |
| Lactobacillus obligate | 1 | 2 | 16 | 16 | 16 | 1 | 1 | 4 | 4 |
| Homofermentative | | | | | | | | | |
| L. gasseri DS108-B6 | 0.25 | 1 | 32 | >256 | 16 | 1 | 2 | 2 | 4 |
| Lactobacillus paracasei | 4 | n.r | 32 | 64 | 64 | 1 | 1 | 4 | 4 |
| L. paracasei DS108-B10 | 0.38 | — | 48 | >256 | 128 | 0.38 | 0.19 | 0.5 | 4 |

As a result, as described in Table 6 above, the DS108-B7 strain of the present invention was confirmed to be below the standard value of EFSA GUARD for ampicillin, vancomycin, erythromycin, clindamycin, and chloramphenicol, and the DS109-B11 strain of the present invention was confirmed to be less than or equal to the standard value of EFSA GUARD for ampicillin, vancomycin, gentamicin, streptomycin, erythromycin, tetracycline, and chloramphenicol. In addition, the DS108-B6 strain of the present invention was confirmed to be below the standard value of EFSA GUARD for r ampicillin, vancomycin, streptomycin, and tetracycline, and the DS108-B10 strain of the present invention was confirmed to be less than or equal to the standard value of EFSA GUARD for ampicillin, erythromycin, clindamycin, tetracycline, and chloramphenicol. Therefore, the four strains of the present invention have low the antibiotic resistance and do not horizontally transfer resistance genes to harmful bacteria present in the intestines. Therefore, there is no concern that harmful bacteria in the intestines will acquire exogenous resistance and cause the antibiotic resistance, making them advantageous for commercialization as the probiotics.

### 2-5. Determination of Intestinal Adhesion Ability

To evaluate the intestinal adhesion ability of the four strains of the present invention, the Caco-2 cell line was used. Specifically, the Caco-2 cell line was cultured in MEM medium containing 10% FBS and 1% penicillin-streptomycin at 37° C. in a 5% CO₂ incubator (PHC, MCO-230AIC, Indonesia). The cells were seeded into 24-well plates at a density of 1×105 cells/well and cultured for 14 days. Then, the cultured cells were suspended in MEM medium to adjust the final concentration to 1×108 CFU/ml, inoculated into each well at 1 ml, and cultured for 2 hours. In this case, glucose was used as the carbon source for each strain. Thereafter, the cultured strains were washed 5 times with PBS (Phosphate Buffered Saline) solution, treated with 0.5% trypsin-EDTA to detach non-attached Caco-2 cells from the plate, diluted with PBS and plated on MRS plate medium, and the viable cell count after 24 hours was measured to evaluate the intestinal adhesion ability of the strains of the present invention. In this case, Bifidobacterium longum DSP19 was used as a control for Bifidobacterium animalis spp. lactis strains.

As a result, as shown in Table 7, when glucose was used as the carbon source, the DS109-B11 strain of the present invention showed the intestinal adhesion rate of about 5.76%, and the DS108-B7 strain showed the intestinal adhesion rate of about 2.54%, confirming that they have intestinal adhesion ability. In addition, the DS108-B10 strain of the present invention showed the intestinal adhesion rate of about 0.66%, and the DS108-B6 strain showed the intestinal adhesion rate of about 1.14%, confirming that they have intestinal adhesion ability.

TABLE 7

| | | | Standard |
|---|---|---|---|
| | | Adhesion | deviation |
| | Strain name | ability(%) | (SD) |
| 1 | Bifidobacterium | 5.76** | 0.754 |
| | animalis | | |
| | spp lactis | | |
| | DS109-B11 | | |
| 2 | Bifidobacterium | 2.54* | 0.371 |
| | animalis | | |
| | spp. lactis | | |
| | DS108-B7 | | |
| 3 | Lactobacillus | 0.66 | 0.227 |
| | paracasei | | |
| | DS108-B10 | | |
| 4 | Lactobacillus | 1.14*** | 0.029 |
| | gasseri | | |
| | DS108-B6 | | |
| Control | B. longum | 0.27* | 0.662 |
| strain | R0175 | | |

*p < 0.5;
**p < 0.05;
***p < 0.001

### Example 2

### Confirmation of AMPK Activation-Promoting Effect of Novel Strains of the Present Invention

The novel strains isolated and identified in Example 1 were confirmed to have AMPK activating activity in muscle cells.

To determine the effects of the novel Bifidobacterium animalis spp. lactis strains, Lactobacillus paracasei strain, or Lactobacillus gasseri strain of the present invention on AMPK protein activity, mouse muscle cell C2C12 (ATCC, American Type Culture Collection), a cell line widely used in the study of muscle differentiation and various intramuscular signaling mechanisms, was treated with the culture medium of the novel Bifidobacterium animalis spp. lactis strains, Lactobacillus paracasei strain, and Lactobacillus gasseri strain of the present invention, and the degree of AMPK protein activity was measured. As a positive control to compare the AMPK protein activity, AICAR (5-aminoimidazole-4-carboxamide ribonucleotide), known as an AMPK activator, was used. The degree of activation of AMPK was measured by quantifying the phosphorylated AMPK protein through Western blotting as AMPK was activated in muscle cells. The amount of the protein was quantified using the ImageJ program as the area of the band generated from the Western blotting results. In addition, for the DS109-B11 strain of the present invention, the efficacy of the AMPK activity was additionally verified using multiple isogenic strains (DS0339, DS0405, and DS0963) as negative controls.

As a result, as illustrated in FIGS. 1 and 2, treatment with the novel Bifidobacterium animalis spp. lactis strains, Lactobacillus paracasei strain, and Lactobacillus gasseri strain of the present invention significantly increased phosphorylation of activated AMPK protein compared to the positive control group.

### Example 3

### Confirmation of Muscle Cell Differentiation

Enhancement Effect of the Novel Strains of the Present

Invention 3-1. In Vitro Effect Confirmation

The novel strains isolated and identified in Example 1 were confirmed to have muscle cell differentiation enhancing activity.

To determine the effects of the novel Bifidobacterium animalis spp. lactis DS109-B11 strain and the Lactobacillus gasseri strain of the present invention on muscle cell differentiation, mouse muscle cell C2C12 cells were treated with the culture medium of the novel Bifidobacterium animalis spp. lactis strain and the Lactobacillus gasseri strain of the present invention, respectively, as described in Example 2. The degree of muscle cell differentiation before and after culture medium treatment was visually compared. Muscle cells were stained using Eosin staining, and the stained area was measured.

As illustrated in FIG. 3, the Eosin-stained area was confirmed to increase in the muscle cell line treated with the culture medium of the Bifidobacterium animalis spp. lactis strain of the present invention. In addition, as illustrated in FIG. 4, the Eosin-stained area was confirmed to increase in the muscle cell line treated with the culture medium of the Lactobacillus gasseri strain of the present invention. According to these results, it was confirmed that muscle cell differentiation was significantly increased by treating with the strain culture medium of the present invention.

### 3-2. In Vivo Effect Confirmation

The efficacy of the DS109-B11 strain of the present invention in improving muscle function was confirmed through in vivo experiments. Specifically, a 1/10 dilution of the culture medium of the DS109-B11 strain was administered orally daily to 6-week-old and 24-month-old mice for 1 to 6 weeks. Grip strength, body weight (bw), muscle weight, and exercise capacity (running distance) of the mouse models were measured. In addition, after treating aged mice with the DS109-B11 strain, the AMPK protein activity and the amount of representative proteins of the mitochondrial complexes I, II, III, IV, and V, which are biomarkers of muscle exercise performance, were analyzed in the aged mouse muscles. The DS0405 strain, which is a strain identical to the DS109-B11 strain of the present invention, served as a control.

As a result, as illustrated in FIG. 5, it was confirmed that treatment with the strain of the present invention increased grip strength and exercise capacity in both young and aged mice compared to the control or untreated groups. It was confirmed that the strain of the present invention may improve the function of both young and aged muscles.

In addition, as illustrated in FIG. 6, treatment with the strain of the present invention increased the AMPK protein activity in aged mouse muscles, similar to in vitro results. This demonstrated that the strain of the present invention improves muscle function by increasing muscle the AMPK activity, not only at the cellular level but also at the animal level.

In addition, as illustrated in FIG. 7, treatment with the strain of the present invention increased the mitochondrial complexes III and IV in aged mouse muscles. Mitochondria serves as a powerhouse, generating oxygen and ATP through a series of redox reactions involving protein complexes I to V, enabling muscle contraction relaxation. These and experimental results demonstrated that treatment with the strain of the present invention may increase mitochondrial quantity in aged muscle, improving muscle function and enhancing exercise performance.

### Preparation Example

### Preparation of Composition Containing Novel Strains of the Present Invention

A composition containing the novel Bifidobacterium animalis spp. lactis strains, Lactobacillus paracasei strain, and Lactobacillus gasseri strain of the present invention was prepared.

The novel Bifidobacterium animalis spp. lactis strains, Lactobacillus paracasei strain, and Lactobacillus gasseri strain of the present invention were each cultured using optimized enrichment media and culture conditions. After recovering a cell mass, 5 to 50% (vol/wt) maltodextrin, 5 to 50% (vol/wt) trehalose, or 5 to 50% (vol/wt) cellulose as cryoprotectants were added relative to the concentrate, followed by freeze-drying and pulverization to produce a lactic acid bacteria powder.

While the above describes representative embodiments of this application, the scope of this application is not limited to the specific embodiments described above. Those skilled in the art will be able to make appropriate modifications within the scope of the claims of this application.

### [Deposit Accession Number]

Depositing Organization: Korea Research Institute of Bioscience and Biotechnology, Biological Resource Center (KCTC)

Deposit accession Number: KCTC15297BP

Date of Deposit: 2023 Jan. 18

Depositing Organization: Korea Research Institute of Bioscience and Biotechnology, Biological Resource Center (KCTC) Deposit accession Number: KCTC15298BP

Date of Deposit: 2023 Jan. 18

Depositing Organization: Korea Research Institute of Bioscience and Biotechnology, Biological Resource Center (KCTC)

Deposit accession Number: KCTC15343BP

Date of Deposit: 2023 Mar. 10

Depositing Organization: Korea Research Institute of Bioscience and Biotechnology, Biological Resource Center (KCTC)

Deposit accession Number: KCTC15299BP Date of Deposit: 2023 Jan. 18

1. An microorganism selected from the group consisting of:
Bifidobacterium animalis spp. lactis DS109-B11 strain, deposited under accession No. KCTC 15297BP,
Bifidobacterium animalis spp. lactis DS108-B7 strain, deposited under accession No. KCTC 15298BP,
Lactobacillus paracasei DS108-B10 strain, deposited under accession No. KCTC 15343BP, and
Lactobacillus gasseri DS108-B6 strain, deposited under accession No. KCTC 15299BP.

2-4. (canceled)

5. A composition comprising one or more of the microorganism according to claim 1, a culture medium of the strain or the combination of the strains, a concentrate of the culture medium, a dried product of the culture medium, and an extract of the culture medium.

6. The composition of claim 5, wherein the composition enhances AMPK activity in a muscle cell.

7. The composition of claim 5, wherein the composition enhances muscle cell differentiation.

8. The composition of claim 5, wherein the composition increases a surface area of muscle.

9. The composition of claim 5, wherein the composition increases mitochondrial synthesis.

10. The composition of claim 5, wherein the composition is a food composition for improving muscle function or a health functional food composition for improving muscle function.

11. The composition of claim 10, wherein the improvement of the muscle function is an enhancement of muscle exercise performance.

12. The composition of claim 5, wherein the composition is a pharmaceutical composition for preventing or treating a muscle disease.

13. The composition of claim 12, wherein the muscle disease is a muscle development disorder, muscle hypokinesia, or sarcopenia.

14. A method for preventing or treating a muscle disease, comprising administering the microorganism according to claim 1 to a subject in need thereof.

15. The method according to claim 14, wherein the muscle disease is a muscle development disorder, muscle hypokinesia, or sarcopenia.

16. The method according to claim 14, wherein the subject requires enhancing AMPK activity in a muscle cell.

17. The method according to claim 14, wherein the subject requires enhancing muscle cell differentiation.

18. The method according to claim 14, wherein the subject requires increasing a surface area of muscle.

19. The method according to claim 14, wherein the subject requires increasing mitochondrial synthesis.
